(19) United States
(12) Patent Application Publication
(10) Pub. No.: US 2011/0004240 A1
(43) Pub. Date: Jan. 6, 2011

(54) VISIBLE LIGHT CURABLE ADHESIVE

(75) Inventors: Yoshihiro Ito, Saitama (JP); Tae-il Son, Saitama (JP); Yusuke Tajima, Saitama (JP)

Correspondence Address: BIRCH STEWART KOLASCH & BIRCH, PO BOX 747, FALLS CHURCH, VA, 22040-0747, US

(73) Assignee: Riken, Saitama (JP)

(21) Appl. No.: 12/866,015

(22) PCT Filed: Feb. 6, 2009
(86) PCT No.: PCT/JP2009/052444

§ 371 Date: Aug. 3, 2010

(30) Foreign Application Priority Data
Feb. 7, 2008 (JP) 2008-027930

(51) Int. Cl. A61B 17/03; C08F 2/50

(52) U.S. Cl. 606/214; 522/63

(57) ABSTRACT

An object of the present invention is to provide a water soluble bioadhesive that is curable by visible light irradiation. The present invention provides a visible light curable adhesive, which comprises a water soluble oxygen sensitizer that generates radicals under visible light irradiation and a water soluble polymer compound having a functional group that can be activated via singlet oxygen generated from the oxygen sensitizer under visible light irradiation.

This application is the national stage application of International Application PCT/JP2009/052444, filed on Feb. 6, 2009, which claims priority under 35 USC §119(a)-(d) of Japanese Application No. JP2008-027930, filed on Feb. 7, 2008.

### TECHNICAL FIELD

The present invention relates to a visible light curable adhesive that can be used for adhesive bonding of living tissues and the like.

### BACKGROUND ART

Hemorrhage during surgery can be treated by appropriate suture or the like in most cases, but hemostasis therefor is often difficult, because of clotting disorder, antithrombotic drugs, inflammation, infection, strong adhesion, or the like (Ono, K., Ishihara, M., Ozeki, Y., Deguchi, H., Sato, M., Saito, Y., Yura, H., Sato, M., Kikuchi, M., Kurita, A., and Maehara, T. (2001) Surgery 130, 844-850). Also, in the field of respiratory surgery, particularly in cases associated with pulmonary emphysema, it is difficult to stop air leakage from the lungs (Ishihara, M., Nakanishi, K., Ono, K., Sato, M., Kikuchi, M., Saito, Y., Yura, H., Matsui, T., Hattori, H., Uenoyama, M., and Kurita, A. (2002) Biomaterials 23, 833-840). To stop such uncontrollable hemorrhage or air leakage from the lungs, materials such as cross-linking gelatin (Ishihara, M., Ono, K., Sato, M., Nakanishi, K., Saito, Y., Yura, H., Matsui, T., Hattori, H., Kikuchi, M., and Kurita, A. (2001) Wound Rep. Reg., 9, 513-521; Braunwald, N. S., Gay, W., and Tatooles, C. J. (1966) Surgery 59, 1024-1030; and Koehnlein, H. E. and Lemperle, G. (1966) Surgery 66, 366-382), cyanoacrylate (Otani, Y., Tabata, Y., and Ikada, Y. (1996) J. Biomed. Mater. Res. 31, 157-166; and Tseng, Y. C., Hyon, S. H., and Ikada, Y. (1990) Biomaterials 11, 73-79), and fibrin glue (Vanholder, R., Misotten, A., Roels, H., and Matton, G. (1993) Biomaterials 14, 737-743; Thetter, O. (1981) Thorac. Cardiovasc. Surg. 29, 290-293; Borst, H. G., Haverish, A., Walterbusch, G., Maatz, W., and Messmer, B. (1982) J. Thorac. Cardiovasc. Surg. 84, 548-553; John, A., Rousou, M. D., Richard, M., Engelman, M. D., and Breyer, R. H. (1984) Ann. Thorac. Surg. 38, 409-410; and Moy, O. J., Peimer, C. A., Koniuchi, M. P., Hoeard, C., Zielezny, M., and Katikaneni, P. R. (1988) J. Hand Surg. 13A, 273-278) have been developed and used.

Tissue adhesives are required to have, in addition to appropriate flexibility and biodegradability, topical or systemic nonirritating properties and nontoxic properties. Cross-linking gelatin and cyanoacrylate have strong tissue adhesive properties. However, it has been discovered that cytotoxicity or strong tissue irritating properties are exerted by aldehyde, imide, or the like, which is generated upon a crosslinking reaction of gelatin or a degradation reaction of cyanoacrylate.

Currently, the most broadly used bioadhesive is fibrin glue comprising fibrinogen, thrombin, XIII factor, and a protease inhibitor, to which the blood coagulation system is applied. The effectiveness of fibrin glue as a bioadhesive has been widely recognized, such as in terms of hemostasis and the blocking of air leakage. However, fibrin glue is problematic in that it is difficult to ensure a sufficient supply of raw materials, since it is a human blood product. Moreover, fibrin glue is a blood product, so that the risk of infection cannot be denied. Issues concerning drug-induced hepatitis C virus arise from such problem. Therefore, various bioadhesives are under development (Wang, D.-A., Varghese, S., Sharma, B., Strehin, I., Fermanian, S., Gorham, J., Fairbrother, D. H., Cascio, B., Elisseeff, J. H. Nat. Mater., 6, 385 (2007); and Mooney, D. J. and Silv, E. A. Nat. Mater., 6, 327-328 (2007)).

Under such circumstances, UV (ultraviolet)-curable bioadhesives have been reported (Ishihara, M. Trends in Glycoscience and Glycotechnology, 14, 331-341 (2002); JP Patent Publication (Kokai) No. 2001-224677 A; and JP Patent Publication (Kokai) No. H6-73102 A (1994)). However, ultraviolet irradiation poses the risk of having severe effects on tissues. Regarding dental materials, resins curable by UV irradiation were used about 20 years ago. Since then, such resins have been replaced by visible light curable resins. If a visible light curable bioadhesive as a soft tissue adhesive to be used in surgery, orthopedics, or dentistry can be realized, it can be expected that such visible light curable bioadhesive will make a great contribution to medical care.

JP Patent Publication (Kokai) No. H10-90893 A (1998) discloses a photosensitive resin composition curable by visible light irradiation that is not intended to be used for adhesives, but contains fullerene and a polymer compound having a functional group that can react with fullerene under visible light irradiation.

### DISCLOSURE OF THE INVENTION

The composition disclosed in JP Patent Publication (Kokai) No. H10-90893 A (1998) is problematic in that since the composition contains fullerene used herein, it is dissolved in only an organic solvent and cannot be used as a bioadhesive.

An object of the present invention is to provide a water soluble bioadhesive that is curable by visible light irradiation.

The present invention encompasses the following (1) to (7).
(1) A visible light curable adhesive, which comprises
a water soluble oxygen sensitizer that is excited under visible light irradiation and
a water soluble polymer compound having a functional group that can be activated via singlet oxygen generated from the oxygen sensitizer under visible light irradiation.
(2) The visible light curable adhesive according to (1), which further contains water.
(3) The visible light curable adhesive according to (1) or (2), which is used for adhesive bonding of living tissues.
(4) The visible light curable adhesive according to any one of (1) to (3), wherein the oxygen sensitizer is rose bengal.
(5) The visible light curable adhesive according to any one of (1) to (4), wherein the functional group has a conjugated diene structure.
(6) The visible light curable adhesive according to (5), wherein the functional group is a furan group.
(7) A method for adhesive bonding of living tissues, comprising the steps of:
applying the visible light curable adhesive according to any one of (1) to (6) between living
tissues to be adhered to each other; and
curing the adhesive by visible light irradiation.

This description includes part or all of the contents as disclosed in the descriptions and/or drawings of Japanese Patent Application No. 2008-027930, which is a priority document of the present application.

### BRIEF DESCRIPTION OF THE DRAWINGS

FIG. 1 shows the results of adding 1% rose bengal and then performing visible light irradiation.

FIG. 2 shows the results of adding 3% rose bengal and then performing visible light irradiation.

FIG. 3 shows a photo of the surface of pig skin cured and adhered using the adhesive composition of the present invention.

FIG. 4 shows a photo of the surface of pig skin cured and adhered using the adhesive composition of the present invention.

### PREFERRED EMBODIMENTS OF THE INVENTION

### 1. Application of Adhesive

The visible light curable adhesive of the present invention can be used for causing organic materials to adhere to each other. The visible light curable adhesive of the present invention allows curing by visible light irradiation, so that the adhesive can be appropriately used for adhesive bonding of living tissues. The visible light curable adhesive of the present invention is useful as an adhesive for soft tissue in surgery, orthopedics, or dentistry.

### 2. Oxygen Sensitizer

An oxygen sensitizer (also referred to as a photosensitizer) to be used in the present invention is not particularly limited, as long as it is a water soluble oxygen sensitizer that is excited under irradiation with visible light, and preferably visible light with a wavelength ranging from 400 nm to 700 nm. Examples of such oxygen sensitizer include rose bengal, methylene blue, bromphenol blue, eosin, and erythrosine.

### 3. Functional Group

A water soluble polymer compound to be used in the present invention has a functional group that can be activated via singlet oxygen generated from the oxygen sensitizer under visible light irradiation.

As such functional group, specifically, a functional group having a conjugated diene structure is preferred, such as a furan group, a thiophene group, or a pyrrole group. Also, functional groups containing primary amines, secondary amines, tertiary amines, anthracenes, enones, benzoins, or the like can also be appropriately used as “functional groups that can be activated via singlet oxygen generated from the oxygen sensitizer under visible light irradiation.”

### 4. Polymer Compound

A polymer compound to be used in the present invention has at least one functional group described above in one molecule and is water soluble.

As a water soluble polymer compound, a biodegradable synthetic polymer can be appropriately used in addition to a protein (e.g., gelatin), or a biopolymer compound such as a polysaccharide. If a polymer compound is biodegradable, the adhesive of the present invention is degraded in vivo.

A functional group can be introduced into a polymer compound by a general method. For example, a furan group can be introduced by reacting furfuryl isocyanate with a polymer compound. The proportion of a functional group to a polymer compound is not particularly limited. Preferably, a functional group accounts for an amount ranging from 0.5% by weight to 20% by weight of a polymer compound after functional group introduction.

### 5. Composition of Visible Light Curable Adhesive

The visible light curable adhesive of the present invention preferably contains the above oxygen sensitizer and the polymer compound at a weight ratio ranging from 1:100 to 20:100.

The visible light curable adhesive of the present invention is preferably provided in a form of an aqueous solution in which the above oxygen sensitizer and the polymer compound are dissolved in water. Water may be used in the form of physiological saline such as a physiological salt solution or Ringer's solution or balanced saline such as phosphate buffer. When the adhesive composition of the present invention is in the form of an aqueous solution, the concentration of an oxygen sensitizer preferably ranges from 0.1% by weight to 20% by weight, and the concentration of a polymer compound preferably ranges from 0.1% by weight to 50% by weight with respect to the total amount of the adhesive composition.

### 6. Usage

The visible light curable adhesive of the present invention is arranged between organic materials (e.g., living tissues) to be adhered to each other, and then light with a wavelength ranging from 400 nm to 700 nm is irradiated from a visible light source, so that adhesion can be performed.

The present invention provides a method for adhesive bonding of living tissues, comprising the steps of: applying the above visible light curable adhesive composition between living tissues to be adhered to each other; and irradiating visible light to cure the adhesive composition. Here, the term “living tissues” refers not only to tissues such as the original skin of organisms and the like, but also to living tissues implanted from outside. Such living tissues are preferably soft tissues such as skin.

### Example 1

### 1. Preparation of Gelatin into which a Furan Group is Introduced

Two (2) g of gelatin was dissolved in 189 mL of pure water at 40° C. and then the pH was adjusted to 9 using dilute aqueous sodium hydroxide. To the solution, a solution prepared by dissolving 300 μL of furfuryl isocyanate in 20 mL of dimethyl sulfoxide was added dropwise in an ice bath. The color of the aqueous solution changed from light yellow to thick yellow. Subsequently, the solution was left to stand overnight at room temperature. Finally, after 2 hours of reaction at 40° C., neutralization was performed with dilute hydrochloric acid, and then dialysis was performed for 2 days, followed by freeze-drying. It was considered that furfuryl isocyanate could be introduced into about 80% of amino groups such as lysine side chains.

### 2. Curing Reaction

The gelatin (0.053 g) into which a furan group had been introduced was dissolved in 1.7 mL of pure water at 40° C. A predetermined amount of rose bengal was dissolved in this solution. The solution was applied to a glass plate in an amount of 30 mL per plate and then irradiation was performed using a visible light irradiator for dentistry (Jetlite3000 J. Morita, U.S.A. Inc.).

Photocuring degree was measured by the following procedures. A glass plate to which the solution had been applied as described above was placed upright after light irradiation for a predetermined time, and then the distance that the applied solution flowed was measured. In this manner, the distance of a solution to flow was measured for samples differing in light irradiation time. The distance moved by a solution of a sample exposed to irradiation for light irradiation time “t” is designated “X.” The distance “Y” that an unexposed sample flowed was determined to be the distance moved at the photocuring degree of 0%. The solution completely stopped flowing after a certain period of time of light irradiation. Accordingly, the distance at the photocuring degree of 100% is zero. The photocuring degree of a sample exposed to irradiation for light irradiation time “t” was calculated by the following formula.

Photocuring degree (%) in a sample exposed to irradiation for light irradiation time “t”=100×(Y−X)/Y

### 3. Results

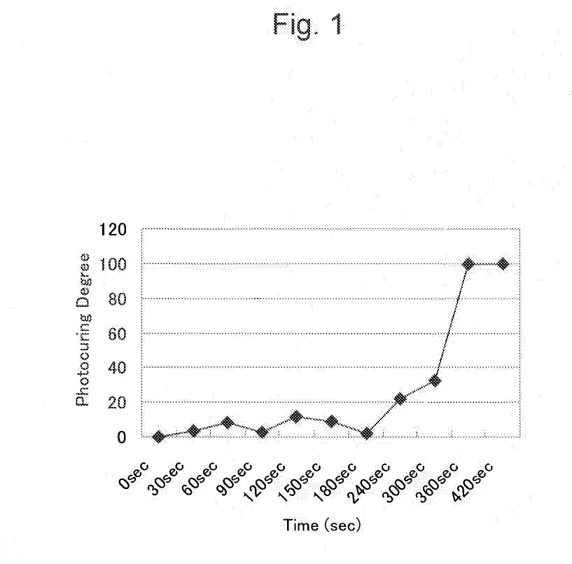

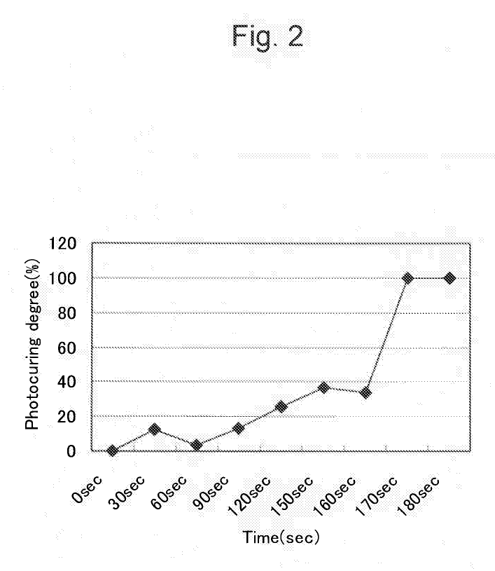

FIG. 1 shows the results of adding 1% rose bengal and then performing visible light irradiation. Curing was found to take place within about 6 minutes. FIG. 2 shows the results of adding 3% rose bengal and then performing visible light irradiation. In this case, curing was observed within about 3 minutes. No curing was observed at all in cases in which no rose bengal had been present and in cases in which visible light irradiation had been performed after addition of rose bengal to unmodified gelatin. It was considered that a furan group introduced into gelatin had formed a radical because of the presence of rose bengal as an oxygen sensitizer, following which crosslinking reaction had taken place by the photooxidation-induced crosslinking mechanism.

### Example 2

An aqueous solution containing a 10 wt % gelatin derivative and 1.0 wt % rose bengal was prepared as an adhesive composition. The gelatin derivative prepared in Example 1 was used.

An incision was made in the center of a section of pig skin (1 cm×2 cm).

The above adhesive composition was applied to the incision on the pig skin, followed by 15 minutes of irradiation using a JETLITE3000 irradiator.

### Conditions:

JETLITE3000 (400 nm-520 nm, 400 mW/cm²) Irradiation distance: 5 mm.

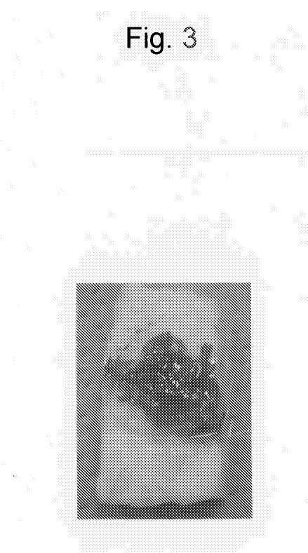

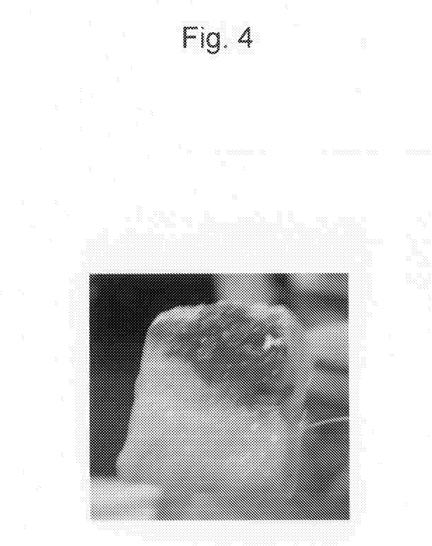

After irradiation, curing was observed on the surface of the pig skin (see FIG. 3 and FIG. 4). When the adhesive composition was applied to an incision on the pig skin, the adhesive composition was firmly cured within 15 minutes, so that the incision could not be opened when both ends were bent or stretched. It was confirmed that the product cross-linked by light irradiation of the adhesive composition was not dissolved in water, but firmly adhered to the material to be adhered, although it contained water to be softened.

### INDUSTRIAL APPLICABILITY

The present invention provides a water soluble adhesive that is curable by visible light irradiation. The adhesive of the present invention is particularly useful for adhesive bonding of living tissues.

All publications, patents, and patent applications cited herein are incorporated herein by reference in their entirety.

1. A visible light curable adhesive, which comprises
a water soluble oxygen sensitizer that is excited under visible light irradiation and
a water soluble polymer compound having a functional group that can be activated via singlet oxygen generated from the oxygen sensitizer under visible light irradiation.

2. The visible light curable adhesive according to claim 1, which further contains water.

3. The visible light curable adhesive according to claim 1, which is used for adhesive bonding of living tissues.

4. The visible light curable adhesive according to claim 1, wherein the oxygen sensitizer is rose bengal.

5. The visible light curable adhesive according to claim 1, wherein the functional group has a conjugated diene structure.

6. The visible light curable adhesive according to claim 5, wherein the functional group is a furan group.

7. A method for adhesive bonding of living tissues, comprising the steps of:
applying the visible light curable adhesive according to claim 1 between living tissues to be adhered to each other; and
curing the adhesive by visible light irradiation.
